(19) United States
(12) Patent Application Publication
(10) Pub. No.: US 2015/0231085 A1
(43) Pub. Date: Aug. 20, 2015

(54) PHARMACEUTICAL COMPOSITE CAPSULE FORMULATION COMPRISING IRBESARTAN AND HMG-COA REDUCTASE INHIBITOR

(71) Applicant: HANMI PHARM. CO., LTD., Hwaseong-si, Gyeonggi-do (KR)

(72) Inventors: Yong Il Kim, Suwon-si (KR); Yoeng Jin Kwon, Hwaseong-si (KR); Caleb Hyungmin Park, Suwon-si (KR); Seung Yeop Lee, Suwon-si (KR); Jae Hyun Park, Suwon-si (KR); Jong Soo Woo, Suwon-si (KR)

(73) Assignee: HANMI PHARM. CO., LTD., Hwaseong-si, Gyeonggi-do (KR)

(21) Appl. No.: 14/420,953

(22) PCT Filed: Aug. 30, 2013
(86) PCT No.: PCT/KR2013/007841

§ 371 Date: Feb. 11, 2015

(30) Foreign Application Priority Data
Aug. 31, 2012 (KR) 10-2012-0096036

(51) Int. Cl. A61K 9/48; A61K 31/40; A61K 9/50; A61K 31/4178

(52) U.S. Cl. CPC A61K 9/4808; A61K 31/4178; A61K 31/40; A61K 9/5089

(57) ABSTRACT

Disclosed are a pharmaceutical composite capsule formulation comprising 1) an independent irbesartan unit comprising irbesartan or a pharmaceutically acceptable salt thereof; and 2) an independent HMG-CoA reductase inhibitor unit comprising an HMG-CoA reductase inhibitor or a pharmaceutically acceptable salt thereof, and an alkaline additive, wherein said independent units are separated from each other within a capsule, and a method for preparing the same. Designed to prevent an interaction between irbesartan and the HMG-CoA reductase inhibitor, the pharmaceutical composite capsule formulation is improved in stability and dissolution rate, and thus shows great bioavailability. In addition, the formulation is expected to guarantee high drug compliance owing to its small size, and therefore can be applied to the treatment of hypertension and hypercholesterolemia.

### FIELD OF THE INVENTION

The present invention relates to a pharmaceutical composite capsule formulation, improved in stability and dissolution rate, comprising 1) an independent irbesartan unit comprising irbesartan or a pharmaceutically acceptable salt thereof; and 2) an independent HMG-CoA reductase inhibitor unit comprising an HMG-CoA reductase inhibitor or a pharmaceutically acceptable salt thereof, and an alkaline additive, wherein said independent units are separated from each other within a capsule, and a method for preparing the same.

### BACKGROUND OF THE INVENTION

“Hyperlipidemia” involves abnormally elevated levels of any or all lipids, such as cholesterol or triglycerides, in blood. Hyperlipidemia, particularly hypercholesterolemia, causes aortic thrombosis, inducing the accumulation of lipids along blood vessels, which leads to the onset of arteriosclerosis. In turn, this reduces the flow of blood, which acts as an underlying cause of ischemic heart diseases, angina pectoris and myocardial infarction. Because there is an apparent causational relationship between hyperlipidemia and arteriosclerosis, the treatment of hyperlipidemia makes a great contribution to the prevention of arteriosclerosis.

HMG-CoA reductase inhibitors have been used for the treatment of hyperlipidemia owing to their ability to lower levels of total cholesterol as well as LDL-cholesterol by inhibiting the enzyme HMG-CoA reductase, the key enzyme of the mevalonate pathway that is responsible for the biosynthesis of cholesterol (see Grundy, S. M. et al., N Engl J Med, 319(1): 24-32, 25-26, 31(1998)).

Irbesartan, represented by the compound of formula (I) (IUPAC name: 2-butyl-3-({4-[2-(2H-1,2,3,4-tetrazol-5-yl)phenyl]phenyl}methyl)-1,3-diazaspiro[4.4]non-1-en-4-one, U.S. Pat. No. 5,270,317), is a potent angiotensin II receptor antagonist, which blocks the interaction of angiotensin II, a causative agent of vasoconstriction, with angiotensin II AT₁ receptors to induce a decrease in blood pressure. The compound is selective for AT₁ receptors, but does not block angiotensin II from binding to AT₂ receptors, thus suppressing endothelial cell growth, vasoconstriction and tissue regeneration while allowing the vasodilatation activity. Because the therapeutic effects of such angiotensin II receptor antagonists have been proven in clinical trials, they are now commercially available as drugs for hypertension, and showed rapid progress in the market (see Jessica C. Song Pharm. D., C. Michael White Pharm. D., Pharmacotherapy, 20(2): 130-139, 2000).

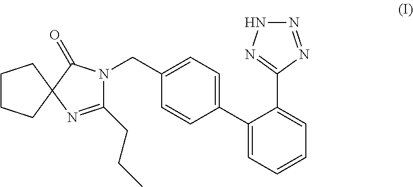 [SMILES: CCCC1=NC2(CCCC2)C(=O)N1Cc1ccc(-c2ccccc2-c2nn[nH]n2)cc1]

As many as approximately 60% of hypertension patients also suffer from hyperlipidemia, and there has been much evidence of close correlation between hypertension and hyperlipidemia. Thus, a combination therapy of an angiotensin II receptor antagonist and an HMG-CoA reductase inhibitor exerts not only a synergistic effect on the treatment of hypertension and hyperlipidemia in patients with cardiovascular diseases, compared to either of the drugs alone, but also a therapeutic effect on diabetes by improving the function of endothelial cells, which form a protective layer of blood vessels, to increase sensitivity to insulin (see Ceriello A, Assaloni R, Da Ros R, Maier A, Piconi L, Quagliaro L, et al., Circulation, 111: 2518-2524, May 2005; and Koh K K, Quon M J, Han S H et al., Circulation, 110: 3687-3692, December 2004).

Korean Patent Laid-Open Publication Nos. 2009-0114328 and 2009-0114190 disclose composite formulations comprising irbesartan and atorvastatin which are designed to release one of the two drugs 2 hours before the sustained release of the other in order to prevent the interaction of the angiotensin receptor block (ARB) drug, irbesartan, with the HMG-CoA reductase inhibitor, atorvastatin. However, the sustained release composite formulation was designed on the basis of in vitro test data. In practice, it is difficult not only to produce a formulation that constantly releases a drug in a sustained manner in vivo, but also to exactly predict the delayed time of release, because gastrointestinal motility differs from one person to another.

Irbesartan is metabolized by the liver via the cytochrome P450 system, predominantly by the 2C9 isozyme. In contrast, an HMG-CoA reductase inhibitor is far less prone to undergoing liver metabolism, but is oxidized primarily by the 3A4 isozyme of cytochrome P450. Considering these circumstances, there is no likelihood of pharmaceutical interaction between irbesartan and an HMG-CoA reductase inhibitor (see Yoshihisa Shitara, Yuichi Sugiyama, Pharmacology & Therapeutics, Vol. 112, Issue 1, October 2006. 71-105, and FDA Avapro label). Accordingly, it is preferred that the two drugs, which are predicted to have no interaction therebetween, be formulated into an immediate release form.

In order to prevent the side effects anticipated upon the co-existence of two or more active ingredients that have physical or chemical interaction therebetween or thereamong, many formulations designed to separate active ingredients from each other or one another, such as two-layer tablets, double layer coated drugs, tablets containing coated pellets, etc., have been suggested. However, such formulations do not guarantee complete separation of active ingredients from each other because of the possibility of contamination by incorporation during manufacturing processes. In the case of two-layer tablets, for example, granules of two active ingredients may be compressed into a tablet while they are incorporated with each other due to various factors of a tableting machine itself including voids, vibration, oscillation and other design problems. Thus, a two-layer tablet has the structural drawback of being unable to perfectly shield active ingredients from each other. A problem with double layer coated drugs is the high likelihood of interlayer contamination due to abrasion and disintegration during a coating process.

Korean Patent Laid-Open Publication No. 2011-0007602 discloses a capsule in a polypill form which comprises a coated tablet of acetylsalicylic acid, a coated tablet of an HMG-CoA reductase inhibitor and a coated tablet of an angiotensin converting enzyme (ACE). However, the number of tablets is limited, and nowhere is an improvement in stability and dissolution mentioned for each ingredient in the invention.

International Patent Publication No. WO 03/011283 discloses a composite formulation comprising atorvastatin calcium and amlodipine besylate in which an alkalizing agent that forms pH of 5 or greater is used as a stabilizer for atorvastatin calcium. However, the alkalizing agent has a negative influence on the stability of the other main ingredient.

There is also a formulation comprising irbesartan and an HMG-CoA reductase inhibitor as separate granules, but the two active ingredients decrease in stability because the contact therebetween cannot be fundamentally avoided. In addition, the formulation comprising the granules is too large in size and volume to be filled in a capsule, or its drug compliance becomes poor.

Leading to the present invention, the present inventors have conducted intensive and thorough research into a composite drug formulation capable of effectively releasing active ingredients, with the aim of solving the problems encountered in the prior art, and developed an immediate release capsule formulation in which irbesartan and an HMG-CoA reductase inhibitor exist separately from each other without interaction therebetween, thereby exhibiting high stability and dissolution profiles.

### SUMMARY OF THE INVENTION

It is an object of the present invention to provide a pharmaceutical formulation comprising irbesartan and an HMG-CoA reductase inhibitor which exhibits an improvement in dissolution and bioavailability of the active ingredients.

It is another object of the present invention to provide a method for preparing the pharmaceutical formulation.

In accordance with an aspect thereof, the present invention provides a pharmaceutical composite capsule formulation comprising: 1) an independent irbesartan unit comprising irbesartan or a pharmaceutically acceptable salt thereof; and 2) an independent HMG-CoA reductase inhibitor unit comprising an HMG-CoA reductase inhibitor or a pharmaceutically acceptable salt thereof, and an alkaline additive, wherein said independent units are separated from each other within a capsule.

In accordance with another aspect thereof, the present invention provides a method for preparing the pharmaceutical composite capsule formulation, comprising: 1) forming irbesartan granules or tablets comprising irbesartan or a pharmaceutically acceptable salt thereof; 2) forming HMG-CoA reductase inhibitor granules or tablets comprising an HMG-CoA reductase inhibitor or a pharmaceutically acceptable salt thereof, and an alkaline additive; and 3) loading the irbesartan granules or tablets of step 1) and the HMG-CoA reductase inhibitor granules or tablets of step 2) into a hard capsule, such that said irbesartan granules or tablets are separated from said HMG-CoA reductase inhibitor granules or tablets within the capsule.

Capable of allowing irbesartan and the HMG-CoA reductase inhibitor to be immediately released while neither generating an interaction therebetween nor causing a sequent decrease in drug dissolution, the composite capsule formulation according to the present invention ensures high dissolution and bioavailability of the active ingredients. In addition, the composite capsule formulation guarantees stability of the active ingredients over time, and is very low in excipient content and thus in formulation size, which leads to an increase in drug compliance.

### BRIEF DESCRIPTION OF THE DRAWINGS

The above and other objects and features of the present invention will become apparent from the following description of the invention, when taken in conjunction with the accompanying drawings.

FIGS. 1 and 2 show degradation products of atorvastatin and irbesartan over time during long-term storage of the formulations of Example 5 and Comparative Examples 1 to 3, respectively;

FIGS. 3 and 4 show dissolution rates of irbesartan and atorvastatin in the formulations of Example 5 and Comparative Examples 1 to 3, respectively;

FIG. 5 shows solubilities of irbesartan of the formulations of Example 5 and Comparative Example 1;

FIG. 6 shows pharmacokinetic parameters of irbesartan in the formulations of Example 5 and Comparative Example 1; and

FIG. 7 shows photographs of the formulation of the present invention.

### DETAILED DESCRIPTION OF THE INVENTION

A detailed description will be given of the present invention, below.

The present invention provides a pharmaceutical composite capsule formulation comprising: 1) an independent irbesartan unit comprising irbesartan or a pharmaceutically acceptable salt thereof; and 2) an independent HMG-CoA reductase inhibitor unit comprising an HMG-CoA reductase inhibitor or a pharmaceutically acceptable salt thereof, and an alkaline additive, wherein said independent units are separated from each other within a capsule. An embodiment of the pharmaceutical composite capsule formulation according to the present invention is shown in FIG. 7.

In the inventive pharmaceutical composite capsule formulation, the independent irbesartan unit and the independent HMG-CoA reductase inhibitor unit are each in a granule or tablet form. At least one of the independent irbesartan unit and the independent HMG-CoA reductase inhibitor unit may take a tablet form. In other words, the capsule formulation may comprise the irbesartan granules or tablets, and the HMG-CoA reductase inhibitor granules or tablets, with the proviso that at least one of the active ingredients is in the form of a tablet.

In one embodiment of the present invention, therefore, the capsule formulation is a hard capsule into which 1) the irbesartan granules or tablets comprising irbesartan or a pharmaceutically acceptable salt thereof; and 2) the HMG-CoA reductase inhibitor granules or tablets comprising an HMG-CoA reductase inhibitor or a pharmaceutically acceptable salt thereof, and an alkaline additive, are loaded while remaining separate from each other. Preferably, the tablet may be a mini-tablet with dimensions of 3 mm or less in both diameter and thickness. Each of the independent units may be coated to ensure a more complete physical shield between them.

According to another embodiment thereof, the present invention provides a capsule formulation in the form of a hard capsule in which tablets comprising irbesartan or a pharmaceutically acceptable salt thereof; and tablets comprising an HMG-CoA reductase inhibitor or a pharmaceutically acceptable salt thereof, and an alkaline additive, are loaded. The capsule formulation may be prepared, for example, by compressing irbesartan or a pharmaceutically acceptable salt thereof into tablets, separately compressing an HMG-CoA reductase inhibitor or a pharmaceutically acceptable salt thereof, together with an alkaline additive, into tablets, and loading both the tablets into a capsule with an appropriate size, e.g., capsule size 1.

In another embodiment, the present invention provides a capsule formulation in the form of a hard capsule in which granules comprising irbesartan or a pharmaceutically acceptable salt thereof; and tablets comprising an HMG-CoA reductase inhibitor or a pharmaceutically acceptable salt thereof, and an alkaline additive, are loaded.

In another embodiment, the present invention provides a capsule formulation in the form of a hard capsule in which tablets comprising irbesartan or a pharmaceutically acceptable salt thereof; and granules comprising an HMG-CoA reductase inhibitor or a pharmaceutically acceptable salt thereof, and an alkaline additive, are loaded.

The independent irbesartan unit according to the present invention comprises irbesartan or a pharmaceutically acceptable salt thereof as an active ingredient. Irbesartan or a pharmaceutically acceptable salt thereof is a potent long-acting angiotensin II receptor antagonist with high affinity for angiotensin II AT₁ receptors. When binding to the receptors, irbesartan blocks the activities of angiotensin including vasoconstriction, the release of aldosterone and the retention of water and sodium in the kidney. With these angiotensin antagonistic activities, irbesartan is applicable to the treatment of cardiovascular diseases, inter alia, hypertension and heart failure. So long as it is readily available to those skilled in the art, any pharmaceutically acceptable salt may be used in the present invention. Examples of the salts include a sodium salt, a potassium salt, a calcium salt, a magnesium salt and an ammonium salt.

The independent irbesartan unit according to the present invention may contain irbesartan or a pharmaceutically acceptable salt in an amount of from about 20 to 70 wt %, based on the total weight of the unit, preferably from about 40 to 70 wt %, and may be contained in the unit formulation form in a therapeutically effective amount, for example, corresponding to 8 to 600 mg of the active ingredient, and preferably, 100 to 200 mg of the active ingredient, per unit formulation, but the content is not limited thereto.

In the present invention, the independent irbesartan unit, for example, the irbesartan granules or tablets, may further comprise a pharmaceutically acceptable additive selected from the group consisting of, but not limited to, a binder, a disintegrant, a lubricant, a diluent, a colorant, an anti-tackifier, a surfactant and a mixture thereof. In addition, the independent irbesartan unit may further comprise a surfactant to improve the hydrophobic property of the irbesartan. When included, the surfactant may enhance aqueous granulation, facilitate the release of tablets after compression and accelerate dissolution of the pharmaceutically active ingredient.

Examples of the binder useful in the present invention include sodium carboxymethyl cellulose, ethyl cellulose, hydroxyethyl cellulose, hydroxypropyl cellulose, hydroxypropyl methyl cellulose, methyl cellulose, gelatin, povidone and a mixture thereof, but are not limited thereto. The binder may be used in an amount of from about 2 to 20 wt %, based on the total weight of the granules or tablets, and preferably in an amount of from about 2 to 10 wt %.

The disintegrant useful in the present invention is selected from the group consisting of corn starch, crospovidone, croscarmellose sodium, carboxymethyl cellulose calcium, sodium starch glycolate, low-substituted hydroxypropyl cellulose and a mixture thereof, but is not limited thereto. The disintegrant may be used in an amount of from about 1 to 20 wt %, based on the total weight of the granules or tablets, and preferably from about 1 to 15 wt %.

The lubricant useful in the present invention may be selected from the group consisting of calcium stearate, glyceryl monostearate, glyceryl palmitostearate, magnesium stearate, sodium lauryl sulfate, sodium stearyl fumarate, zinc stearate, stearic acid, hydrogenated vegetable oil, polyethylene glycol, sodium benzoate, talc and a mixture thereof, but is not limited thereto. The lubricant may be used in an amount of from about 0.2 to 5 wt % based on the total weight of the granules or tablets, and preferably in an amount of about 0.5 to 4 wt %.

Examples of the surfactant useful in the present invention include, but are not limited to, sodium lauryl sulfate, a poloxamer, polyethylene glycol and a mixture thereof, with preference for a poloxamer. It is preferred that the surfactant be contained only in the independent irbesartan unit in view of stability, but may be in the other independent unit.

According to one preferred embodiment, the independent irbesartan unit may comprise (a) irbesartan in an amount of from 20 to 70 wt % (e.g., 50 wt %), (b) a diluent in an amount of from 1 to 70 wt %, (c) a binder in an amount of from 2 to 20 wt %, (d) a disintegrant in an amount of from 1 to 20 wt %, (e) an anti-tackifier in an amount of from 0.1 to 5 wt %, (f) a lubricant in an amount of from 0.2 to 5 wt %, and (g) a colorant in an amount of less than 2 wt % (e.g., 0.1 to 1 wt %), based on the total weight of the irbesartan granules or tablets.

Meanwhile, the independent HMG-CoA reductase inhibitor unit comprises an HMG-CoA reductase inhibitor or a pharmaceutically acceptable salt thereof, and an alkaline additive.

In the present invention, the HMG-CoA reductase inhibitor may be selected from the group consisting of rosuvastatin (U.S. Pat. No. 4,231,938), lovastatin, atorvastatin, pravastatin (U.S. Pat. Nos. 4,346,227 and 4,410,629), fluvastatin, pitavastatin, simvastatin (U.S. Pat. Nos. 4,448,784 and 4,450,171), rivastatin, cerivastatin, velostatin, mevastatin (U.S. Pat. No. 3,983,140), a pharmaceutically acceptable salt thereof, a precursor thereof and a mixture thereof, preferably atorvastatin calcium, but not limited thereto.

The independent HMG-CoA reductase inhibitor unit according to the present invention may comprise an HMG-CoA reductase inhibitor or a pharmaceutically acceptable salt in an amount of from about 5 to 20 wt %, based on the total weight of the unit, preferably 5 to 10 wt % (e.g., about 8 wt %), and may be contained in the unit formulation form in a therapeutically effective amount, for example, corresponding to 0.5 to 100 mg of the active ingredient, preferably, 2.5 to 80 mg of the active ingredient, and more preferably 5 to 80 mg of the active ingredient, per unit formulation, but not limited thereto.

As described above, the alkaline additive exists only in the HMG-CoA reductase inhibitor unit so as to increase the stability of the HMG-CoA reductase inhibitor. Later, the alkaline additive in the HMG-CoA reductase inhibitor unit also functions to improve the bioavailability of irbesartan by providing an alkaline environment under which irbesartan increases in solubility.

The alkaline additive may be selected from the group consisting of an alkaline inorganic compound (e.g., NaHCO₃, CaCO₃, MgCO₃, KH₂PO₄, K₂HPO₃ and calcium phosphate tribasic), arginine, lysine, histidine, meglumine, aluminum magnesium silicate, aluminum magnesium metasilicate, a salt thereof and a mixture thereof, preferably NaHCO₃, CaCO₃, MgCO₃, or a mixture thereof, but not limited thereto. The alkaline additive may be used in an amount of from 2 to 10 parts by weight, based on 1 part by weight of the HMG-CoA reductase inhibitor, and may be contained in an amount of from about 8 to 65 wt %, based on the total weight of the HMG-CoA reductase inhibitor granules or tablets.

In the present invention, the independent HMG-CoA reductase inhibitor unit, for example, the HMG-CoA reductase inhibitor granules or tablets, may further comprise a pharmaceutically acceptable additive selected from the group consisting of an aqueous diluent, a disintegrant, a binder, a carrier, a filler, a lubricant, a rheology modifier, a crystallization retardant, a solubilizer, a colorant, a pH adjuster, a surfactant, an emulsifier, a coating agent, or a mixture thereof.

The aqueous diluent may be selected from among mannitol, sucrose, lactose, sorbitol, xylitol, glucose and a mixture thereof, but not limited thereto.

Examples of the disintegrant include hydroxypropyl cellulose, crospovidone, sodium starch glycolate and croscarmellose sodium. A suitable selection may be made from among disintegrants that are typically used. Preferable examples of the binder include povidone, copovidone and celluloses. Among the lubricants useful in the present invention are magnesium stearate, sodium stearyl fumarate, talc, glyceryl fatty acid esters and glycerol dibehenate. Any typical lubricant may be used. The coating agent may be polyvinyl alcohol, hydroxypropyl methyl cellulose, methyl cellulose, or ethyl cellulose and may be suitably selected from among typically used coating agents.

In accordance with a preferred embodiment of the present invention, the independent HMG-CoA reductase inhibitor may comprise (a) an HMG-CoA reductase inhibitor in an amount of from 5 to 20 wt %, (b) a pharmaceutically acceptable diluent, a disintegrant and a binder in an amount of from 2 to 70 wt %, (c) a lubricant or a coating agent in an amount of from 0.5 to 2 wt %, and (d) an alkaline additive in an amount of from 8 to 65 wt %, based on the total weight of the HMG-CoA reductase inhibitor granules or tablets.

Each of the tablets responsible for the independent irbesartan unit or the independent HMG-CoA reductase inhibitor unit may further comprise a coating layer. The coating layer is applied to at least one of the independent irbesartan unit and the independent HMG-CoA reductase inhibitor unit to completely separate the units from each other, thus improving the stability and dissolution profile of the active ingredients.

Given the coating, the mini-tablet with a dimension of 3 mm or less in diameter and thickness which is loaded to a capsule can have improved mechanical strength, thus having a positive influence on the subsequent loading process and the quality of the final product. In addition, the coating of the mini-tablet makes a great contribution to the production rate of the final product. For example, the coated mini-tablet with suitable mechanical strength can endure the destructive force generated by the hopper and the delivery pump of a capsule loader machine in which the tablet stays.

For a coating layer of the tablet, a typical polymer may be used as a coating material. For example, it is selected from the group consisting of methyl cellulose, ethyl cellulose, polyvinyl alcohol, polyvinyl pyrrolidone, hydroxyethyl cellulose, hydroxypropyl methyl cellulose, and a mixture thereof, but not limited thereto. The coating material is preferably used in a sufficiently small amount so as to impart an optimal size to the formulation and to effectively prepare the formulation. The coating material may be employed in an amount of from 1 to 20 wt %, based on the total weight of the tablet, and preferably from 2 to 10 wt %.

So long as it is accepted in the art, any hard capsule may be employed in the capsule formulation of the present invention. The hard capsule useful in the present invention may be made of gelatin, hypromellose, pullulan (e.g., NP Caps™, Capsugel), or polyvinyl alcohol.

So long as it is accepted for typical medicines, any capsule size may be employed for the hard capsule available in the capsule formulation of the present invention. In the pharmaceutical field, capsule sizes, i.e., internal volumes of capsules, can be discriminated by accompanying capsule size numbers. For example, a volume of 0.95 mL is denoted by capsule size 00, 0.68 mL by capsule size 0, 0.47 mL by capsule size 1, 0.37 mL by capsule size 2, 0.27 mL by capsule size 3 and 0.20 mL by capsule size 4 (refer to web-page of Suheung Capsule). Although smaller capsule sizes are better for drug compliance, capsules with size 0, 1, 2, 3 or 4 may be used in consideration of the content of the active ingredients loaded thereto. Preferred is a capsule size 1, 2 or 3.

In accordance with another aspect thereof, the present invention provides a method for preparing the pharmaceutical composite capsule formulation, comprising: 1) forming irbesartan granules or tablets comprising irbesartan or a pharmaceutically acceptable salt thereof; 2) forming HMG-CoA reductase inhibitor granules or tablets comprising a HMG-CoA reductase inhibitor or a pharmaceutically acceptable salt thereof, and an alkaline additive; and 3) loading the irbesartan granules or tablets of step 1) and the HMG-CoA reductase inhibitor granules or tablets of step 2) into a hard capsule, said HMG-CoA reductase inhibitor granules or tablets existing separate from said HMG-CoA reductase inhibitor granules or tablets within the capsule.

In one embodiment, the method comprises: i) granulating irbesartan or a pharmaceutically acceptable salt thereof in mixture with a pharmaceutically acceptable additive to form granules, and optionally compressing the granules into tablets; ii) granulating an HMG-CoA reductase inhibitor or a pharmaceutically acceptable salt thereof, and an alkaline additive in mixture of a pharmaceutically acceptable additive to form granules, and optionally compressing the granules into tablets; (and optionally, coating the irbesartan granules or tablets of step i) and the HMG-CoA reductase inhibitor granules or tablets of step ii)); and iii) loading the irbesartan granules or tablets of step i) and the HMG-CoA reductase inhibitor granules or tablets of step ii) into a hard capsule, such that said irbesartan granules or tablets are separated from said HMG-CoA reductase inhibitor granules or tablets within the capsule.

The steps of the preparing method of the present invention may be carried out using typical processes. In step i) or ii), the granules may be compressed into tablets using a tableting machine. Preferably, the tablets have suitable hardness, for example, an average hardness of from 1 to 30 kp. The average hardness may be measured prior to the film coating process. Optionally, the method may further comprise coating the irbesartan tablets of step i) and/or the HMG-CoA reductase inhibitor tablets of step ii) before step iv).

In step iii), the irbesartan granules or tablets, and the HMG-CoA reductase inhibitor granules or tablets, are loaded into a hard capsule while remaining separate from each other within the capsule, with the proviso that at least one of the independent units is in a tablet form.

The capsule formulation prepared according to the method of the present invention may be administered via an oral or sublingual route to prevent or treat a disease selected from the group consisting of hypertension, hypercholesterolemia, hyperlipidemia, myocardial infarction, stroke, a disease requiring angioplasty and chronic stable angina pectoris.

Existing in respective separate forms within the capsule, irbesartan and the HMG-CoA reductase inhibitor in the capsule formulation according to the present invention retain their own integrities fully separately. Hence, with the minimal interaction between the two active ingredients, the capsule formulation of the present invention exhibits excellent product stability, which leads to an increase in therapeutic effect. In addition, the capsule formulation of the present invention does not require a new analysis method for evaluating stability with time, but can be assayed for temporal stability using a conventional analysis method for single formulations.

On the basis of the finding that an alkaline additive serving as a stabilizer for an HMG-CoA reductase inhibitor has an influence on the stability of irbesartan, the pharmaceutical composite capsule formulation comprising irbesartan and an HMG-CoA reductase inhibitor is conceived as an immediate release formulation. In the present invention, the active ingredients are separately granulated to form respective granules which are in turn compressed into independent mini-tablets which are optionally coated before being loaded to a hard capsule. Therefore, the capsule product can be stored for a long period of time owing to the high stability of the active ingredients, and is improved in drug compliance owing to its small size attributed to a very low content of excipients. Further, when irbesartan and the HMG-CoA reductase inhibitor are loaded in the form of mini-tablets into a capsule, it allows the immediate release of the active ingredients without undergoing a low dissolution phenomenon caused by an interaction therebetween (releasing irbesartan or a pharmaceutically acceptable salt thereof, and an HMG-CoA reductase inhibitor or a pharmaceutically acceptable salt thereof, at a rate of 80% or higher within 30 min, and preferably at a rate of 80% within 15 min). Therefore, the pharmaceutical composite capsule formulation of the present invention exhibits improved dissolution rate, and excellent oral bioavailability, thereby guaranteeing a promising therapeutic effect.

Hereinafter, the present invention is described more specifically by the following examples, but these are provided only for illustration purposes and the present invention is not limited thereto.

### Example 1

### Preparation of Capsule Comprising Rosuvastatin Mini-Tablet and Irbesartan Granules (1)

According to the data of the column of Example 1 in Table 1, irbesartan (Hanmi Fine Chemical, Korea), lactose, pregelatinized starch and crospovidone were mixed with one another, added with a liquid binder of hydroxypropyl cellulose (HPC-L, Nisso, Japan) and poloxamer 188 (BASF, Germany) in water, and dried, followed by screening the damp mass through a 30-mesh sieve to give wet granules. Subsequently, the wet granules were finally mixed with talc to prepare irbesartan granules.

Separately, as indicated by the composition of the column of Example 1 in Table 1, rosuvastatin calcium, lactose, crospovidone and sodium hydrogen carbonate were mixed with one another, added with a liquid binder of hydroxypropyl cellulose (HPC-L) and polysorbate 80 (Croda, U.S.A.) in water, and dried, followed by screening the damp matter through a 30-mesh sieve to give wet granules. These wet granules were mixed with croscarmellose sodium and finally with magnesium stearate to prepare rosuvastatin calcium granules. The granules thus obtained were then compressed into mini-tablets which were then coated. For compression, a rotary tableting machine (Sejong, GRC-18) was used to produce tablets with a dimension of 2 mm in both diameter and thickness. Hydroxypropyl methyl cellulose was top-sprayed onto a fluidized bed of the mini-tablets using a fluidized bed coater (Dalton, NQ-160).

The irbesartan granules and the rosuvastatin mini-tablets were taken in respectively predetermined amounts as shown in Table 1, and loaded into a hard capsule size 1 using a capsule filler (GKF-2500, Bosch).

### Example 2

### Preparation of Capsule Comprising Rosuvastatin Mini-Tablet and Irbesartan Granules (2)

According to the data of the column of Example 2 in Table 1, irbesartan (Hanmi Fine Chemical, Korea), lactose, pregelatinized starch and crospovidone were mixed with one another, added with a liquid binder of hydroxypropyl cellulose (HPC-L, Nisso, Japan) and sodium lauryl sulfate in water, and dried, followed by screening the damp mass through a 30-mesh sieve to give wet granules. Subsequently, the wet granules were finally mixed with talc to prepare irbesartan granules.

Separately, as indicated by the composition of the column of Example 2 in Table 1, rosuvastatin calcium, microcrystalline cellulose, crospovidone and sodium hydrogen carbonate were mixed with one another, added with a liquid binder of hydroxypropyl cellulose (HPC-L) and polysorbate 80 (Croda, U.S.A.) in water, and dried, followed by screening the damp matter through a 30-mesh sieve to give wet granules. These wet granules were mixed with croscarmellose sodium and finally with magnesium stearate to prepare rosuvastatin calcium granules. The granules thus obtained were then compressed into mini-tablets which were then coated. For compression, a rotary tableting machine (Sejong, GRC-18) was used to produce tablets with a dimension of 2 mm in both diameter and thickness. Hydroxypropyl methyl cellulose was top-sprayed onto a fluidized bed of the mini-tablets using a fluidized bed coater (Dalton, NQ-160).

The irbesartan granules and the rosuvastatin mini-tablets were taken in respectively predetermined amounts as shown in Table 1, and loaded into a hard capsule size 1 using a capsule filler (GKF-2500, Bosch).

TABLE 1
Components and contents of hard capsules comprising irbesartan granules and rosuvastatin
mini-tablets (unit: mg)

| | | Component | Example 1 | Example 2 |
|---|---|---|---|---|
| Irbesartan | Granulation | Irbesartan | 150 | 150 |
| Granule | | Lactose | 30 | 20 |
| | | Pregelatinized starch | 23 | 23 |
| | | Crospovidone | 12 | 6 |
| | | Hydroxypropyl cellulose | 9 | 6 |
| | | Poloxamer 188 | 12 | — |
| | | Sodium lauryl sulfate | — | 9 |
| | Final Mixing | Talc | 4 | 4.5 |
| | | Total weight | 240 | 218.5 |
| Rosuvastatin | Granulation | Rosuvastatin calcium | 10.4 | 10.4 |
| calcium | | Lactose | 15 | — |
| mini-tablet | | Microcrystalline cellulose | — | 16 |
| | | Crospovidone | 7 | 7 |
| | | NaHCO₃ | 60 | 40 |
| | | Hydroxypropyl cellulose | 4 | 5 |
| | | Polysorbate 80 | 0.6 | 0.6 |
| | Mixing | Croscarmellose sodium | 3 | 3 |
| | Final Mixing | Magnesium stearate | 1.25 | 1.25 |
| | Tableting | | Mini-tablet | Mini-tablet |
| | Coating | Hydroxypropyl methyl cellulose | 2 | 2 |
| | | Total weight | 103.25 | 85.25 |

| | | | |
|---|---|---|---|
| Capsule Loading | Total Weight (exclusive of capsule) | 343.25 | 303.75 |

### Example 3

### Preparation of Capsule Comprising Atorvastatin Granules and Irbesartan Mini-Tablet (1)

According to the data of the column of Example 3 in Table 2, irbesartan (Hanmi Fine Chemical, Korea), mannitol, pregelatinized starch and crospovidone were mixed with one another, added with a liquid binder of povidone (BASF, Germany) and poloxamer 188 (BASF, Germany) in water, and dried, followed by screening the damp mass through a 30-mesh sieve to give wet granules. Subsequently, the wet granules were mixed with mannitol, silicon dioxide and crospovidone and finally with magnesium stearate to prepare irbesartan granules. The granules thus obtained were then compressed into mini-tablets and coated. In this regard, the mini-tablets were prepared into a dimension of 2 mm in diameter and thickness using a rotary tableting machine (Sejong, GRC-18). Hydroxypropyl methyl cellulose was top-sprayed onto the mini-tablets using a fluidized bed coater (Dalton, NQ-160).

Separately, as indicated by the composition of the column of Example 3 in Table 2, atorvastatin calcium (TEVA, India), lactose, croscarmellose sodium and sodium hydrogen carbonate were mixed with one another, added with a liquid binder of povidone and polysorbate 80 (Croda, U.S.A.) in water, and dried, followed by screening the damp matter through a 30-mesh sieve to give wet granules. Subsequently, the wet granules were mixed finally with magnesium stearate to prepare atorvastatin calcium granules.

The irbesartan mini-tablets and the atorvastatin granules were taken in respectively predetermined amounts as shown in Table 2 and loaded into a hard capsule size 1 using a capsule filler (GKF-2500, Bosch).

### Example 4

### Preparation of Capsule Comprising Atorvastatin Granules and Irbesartan Mini-Tablet (2)

According to the data of the column of Example 4 in Table 2, atorvastatin calcium (TEVA, India), lactose, croscarmellose sodium and magnesium carbonate (Tomita, Japan) were mixed with one another, added with a liquid binder of povidone and polysorbate 80 (Croda, U.S.A.) in water, and dried, followed by screening the damp mass through a 30-mesh sieve to give wet granules. Subsequently, the wet granules were mixed finally with magnesium stearate to prepare atorvastatin granules.

The irbesartan mini-tablets prepared in the same manner as in Example 3 and the atorvastatin granules were taken in respectively predetermined amounts as shown in Table 2 and loaded into a hard capsule size 1 using a capsule filler (GKF-2500, Bosch).

TABLE 2
Components and contents of hard capsules comprising irbesartan mini-tablets and
atorvastatin granules (unit: mg)

| | | Component | Example 3 | Example 4 |
|---|---|---|---|---|
| Irbesartan | Granulation | Irbesartan | 150 | 150 |
| mini-tablet | | Mannitol | 15 | 15 |
| | | Pregelatinized starch | 20 | 20 |
| | | Crospovidone | 6 | 6 |
| | | Povidone | 8 | 8 |
| | | Poloxamer 188 | 9 | 9 |
| | Mixing | Mannitol | 28.5 | 28.5 |
| | | Silicon dioxide | 10 | 10 |
| | | Crospovidone | 6 | 6 |
| | Final Mixing | Magnesium stearate | 2.5 | 2.5 |
| | Tableting | | Mini-tablet | Mini-tablet |
| | Coating | Hydroxypropyl methyl cellulose | 2 | 2 |
| | | Total weight | 257 | 257 |
| Atorvastatin | Granulation | Atorvastatin calcium | 10.36 | 10.36 |
| calcium | | Lactose | 20 | 20 |
| granules | | Croscarmellose sodium | 10 | 10 |
| | | Povidone | 5 | 5 |
| | | Polysorbate 80 | 0.6 | 0.6 |
| | | Magnesium carbonate | — | 57 |
| | | NaHCO₃ | 57 | — |
| | Final Mixing | Magnesium stearate | 1.25 | 1.25 |
| | | Total weight | 104.21 | 104.21 |

| | | | |
|---|---|---|---|
| Capsule Loading | Total weight (exclusive of capsule) | 361.21 | 361.21 |

### Example 5

### Preparation of Capsule Comprising Atorvastatin Mini-Tablets and Irbesartan Mini-Tablets

According to the composition given in Table 3, irbesartan (Hanmi Fine Chemical, Korea), mannitol, pregelatinized starch and croscarmellose sodium (DMV International) were mixed with one another, added with a liquid binder of povidone (BASF, Germany) and poloxamer 188 (BASF, Germany) in water, and dried, followed by screening the damp mass through a 30-mesh sieve to give wet granules. Subsequently, the wet granules were mixed with mannitol, silicon dioxide and croscarmellose sodium and finally with magnesium stearate to prepare irbesartan granules.

Separately, as indicated by the composition given in Table 3, atorvastatin calcium (TEVA, India), mannitol, microcrystalline cellulose, croscarmellose sodium and magnesium carbonate (Tomita, Japan) were mixed with one another, added with a liquid binder of HPC and polysorbate 80 (Croda, U.S.A.) in water, and dried, followed by screening the damp matter through a 30-mesh sieve to give wet granules. Subsequently, the wet granules were mixed finally with croscarmellose sodium and magnesium stearate to prepare atorvastatin calcium granules.

The irbesartan granules and atorvastatin calcium granules were compressed respectively into mini-tablets, and coated. In this regard, the mini-tablets were prepared into a dimension of 2 mm in diameter and thickness using a rotary tableting machine (Sejong, GRC-18). Opadry™ II 85F18422 white was top sprayed onto the mini-tablets using a fluidized bed coater (Dalton, NQ-160).

The irbesartan mini-tablets and the atorvastatin mini-tablets were taken in respectively predetermined amounts as shown in Table 3 and loaded into a hard capsule size 1 using a capsule filler (GKF-2500, Bosch).

TABLE 3
Components and contents of hard capsules comprising irbesartan and atorvastatin mini-
tablets (unit: mg)

| | | | | Comparative |
|---|---|---|---|---|
| | | Component | Example 5 | Example 1 |
| Irbesartan | Granulation | Irbesartan | 150 | 150 |
| tablet | | Mannitol | 15 | 15 |
| | | Pregelatinized starch | 23 | 23 |
| | | Croscarmellose sodium | 6 | 6 |
| | | Povidone | 8 | 8 |
| | | Poloxamer 188 | 9 | 9 |
| | Mixing | Mannitol | 28.5 | 28.5 |
| | | Silicon dioxide | 10 | 10 |
| | | Croscarmellose sodium | 6 | 6 |
| | Final Mixing | Magnesium stearate | 2.5 | 2.5 |
| | Tableting | | Mini-tablet | Mini-tablet |
| | Coating | Opadry II 85F18422 white | 4 | 4 |
| | | Total weight | 262 | 262 |
| Atorvastatin | Granulation | Atorvastatin calcium | 10.36 | 10.36 |
| calcium tablet | | Mannitol | 10 | 10 |
| | | Microcrystalline cellulose | 6 | 6 |
| | | Croscarmellose sodium | 7 | 7 |
| | | Magnesium carbonate | 57 | — |
| | | HPC | 5 | 5 |
| | | Polysorbate 80 | 0.6 | 0.6 |
| | Mixing | Croscarmellose sodium | 3 | 3 |
| | Final Mixing | Magnesium stearate | 1.25 | 1.25 |
| | Tableting | | Mini-tablet | Mini-tablet |
| | Coating | Opadry II 85F18422 white | 2 | 2 |
| | | Total weight | 102.21 | 45.21 |

| | | | |
|---|---|---|---|
| Capsule Loading | Total weight (exclusive of capsule) | 364.21 | 307.21 |

### Comparative Example 1

### Preparation of Capsule Comprising Alkaline Additive-Free Atorvastatin and Irbesartan Mini-Tablets

A capsule was prepared in the same manner as in Example 5 using the composition given in Table 3, with the exception that magnesium carbonate as an alkaline additive was not used.

### Comparative Example 2

### Preparation of Hard Capsule Comprising Atorvastatin and Irbesartan Tablets

Irbesartan granules and atorvastatin calcium granules were prepared in the same manner as in Example 5. Using a rotary tableting machine (Sejong, GRC-18), the irbesartan granules were compressed into two tablets while the atorvastatin granules were compressed into one tablet. Each of the tablets was prepared into a size of 5 mm in diameter, which was larger than the mini-tablets prepared in the above Examples.

The tablets were coated with Opadry™ II 85F18422 white using a pan coater (Sejong, SFC-30) before being loaded into a hard capsule size 0 in the same manner as in Example 5.

### Comparative Example 3

### Preparation of Two-Layer Tablet Comprising Atorvastatin and Irbesartan

According to the composition given in Table 4 below, granules were respectively prepared from irbesartan (Hanmi Fine Chemical, Korea) and atorvastatin calcium (TEVA, India) in the same manner as in Example 5. Using a two-layer tablet press, the irbesartan granules and the atorvastatin granules were compressed into respective two-layer tablets. Subsequently, the tablets were coated with Opadry™ II 85F18422 white using a pan coater (Sejong, SFC-30).

TABLE 4
Components and contents of two-layer tablet comprising irbesartan
and atorvastatin (unit: mg)

| | | | Comparative |
|---|---|---|---|
| | | Component | Example 3 |
| Irbesartan | Granulation | Irbesartan | 150 |
| Layer | | Mannitol | 47 |
| | | Pregelatinized starch | 23 |
| | | Croscarmellose sodium | 12 |
| | | Povidone | 8 |
| | | Poloxamer 188 | 9 |
| | Final Mixing | Magnesium stearate | 4 |
| Atorvastatin | Granulation | Atorvastatin calcium | 10.85 |
| Layer | | Lactose | 120 |
| | | Microcrystalline cellulose | 65.6 |
| | | Croscarmellose sodium | 36 |
| | | Magnesium carbonate | 57 |
| | | HPC | 3 |
| | | Polysorbate 80 | 1.2 |
| | Final Mixing | Magnesium Stearate | 3 |

| | | |
|---|---|---|
| Tableting | | Two-layer |
| | | tablet |
| Coating | Opadry II 85F18422 white | 2 |
| | Total weight | 551.65 |

### Test Example 1

### Stability Test of Formulation

The stability of the formulations prepared in Example 5 and Comparative Examples 1 to 3 was assayed by measuring degradation products (RRT 1.81) of atorvastatin after each of the formulations was packed, together with 1 g of silica gel, in an HDPE bottle, and stored under a long-term condition (25° C., 60% RH) for 3, 6, 9, 12, 18, 24 and 36 months.

<Analysis Condition for Related Substance of Atorvastatin>

(1) Detector: UV spectrophotometer (detection wavelength 254 nm)

(2) Column: Stainless steel tube about 4.6 mm in inner diameter and about 250 mm in length, loaded with 5 μm C₁₈, or a similar column (e.g., Kromasil 100-5, C18)

(3) Mobile phase A: acetonitrile/tetrahydrofuran/buffer 1 (31:9:60, v/v)
(buffer 1: 0.05 M NH₄H₂PO₄ (pH 5.0, pH adjusted with ammonia water))
Mobile phase B: acetonitrile/buffer 2 (75:25, v/v),
(buffer 2: buffer 1/THF (60:9, v/v))

(4) Diluent: acetonitrile/tetrahydrofuran/water (60:5:35, v/v)

(5) Injection dose: 10 μL

(6) Temperature: 35° C.

(7) Flow rate: 1.8 mL/min

TABLE 5

| | | | Flow |
|---|---|---|---|
| Time (min) | Mobile phase A (%) | Mobile phase B (%) | rate (mL/min) |
| 0 | 100 | 0 | 1.8 |
| 20 | 100 | 0 | 1.8 |
| 30 | 45 | 55 | 2.0 |
| 40 | 0 | 100 | 2.5 |
| 50 | 0 | 100 | 2.5 |

In addition, degradation products (RRT 0.8) of irbesartan were quantitated after each formulation was stored in an acceleration condition (40° C., 75% RH) for 1, 3 and 6 months.

<Analysis Condition for Related Substance of Irbesartan>

(1) Detector: UV spectrophotometer (detection wavelength 220 nm)

(2) Column: stainless steel tube about 4.6 mm in inner diameter and about 250 mm in length, loaded with 5 μm C₁₈, or a similar column

(3) Mobile phase: acetonitrile/phosphate buffer (60:40, v/v)
(phosphate buffer=a solution of 5.5 mL of phosphoric acid in 1 L of pure water (pH 5.0, pH adjusted with triethylamine))

(4) Diluent: methanol

(5) Injection dose: 15 μL

(6) Temperature: 30° C.

(7) Flow rate: 1.2 mL/min

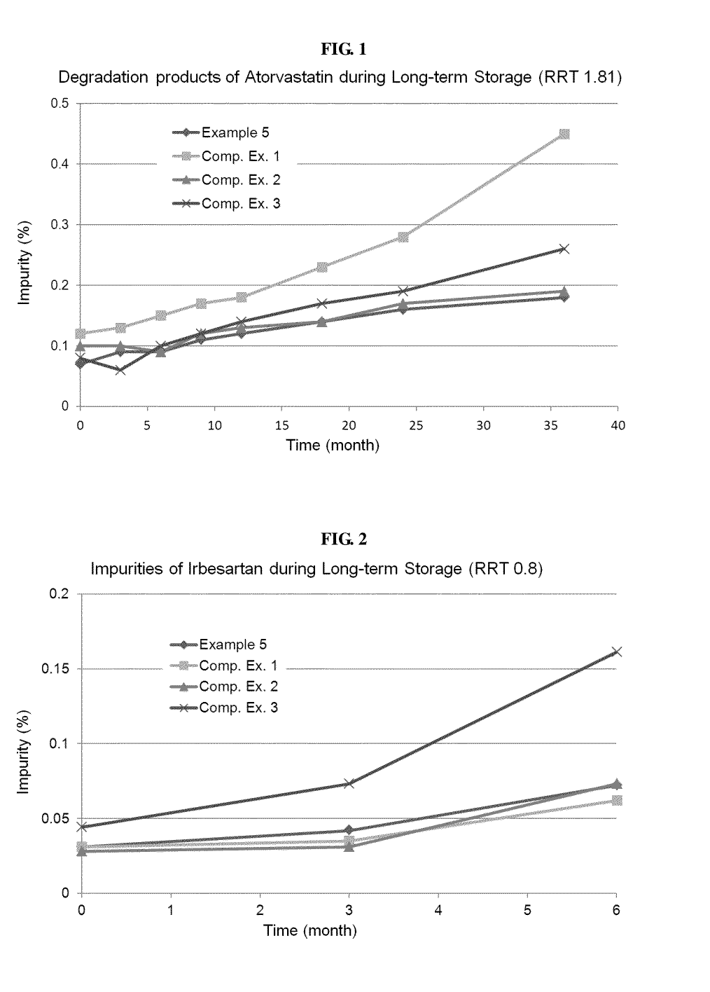

The results are shown in Tables 6A and 6B, and FIGS. 1 and 2.

TABLE 6A
Degradation products of atorvastatin after storage
in long-term condition (25° C., 60% RH) (RRT 1.81)

| | | Long-term condition (months) | | | | | | | |
|---|---|---|---|---|---|---|---|---|---|
| | Example No. | 0 | 3 | 6 | 9 | 12 | 18 | 24 | 36 |
| Degradation | Example 5 | 0.07 | 0.09 | 0.09 | 0.11 | 0.12 | 0.14 | 0.16 | 0.18 |
| product of | Comparative Example 1 | 0.12 | 0.13 | 0.15 | 0.17 | 0.18 | 0.23 | 0.28 | 0.45 |
| atorvastatin | Comparative Example 2 | 0.1 | 0.1 | 0.09 | 0.12 | 0.13 | 0.14 | 0.17 | 0.19 |
| (%) | Comparative Example 3 | 0.08 | 0.06 | 0.1 | 0.12 | 0.14 | 0.17 | 0.19 | 0.26 |

TABLE 6B
Degradation products of atorvastatin after storage in acceleration condition
(40° C., 75% RH) (RRT 0.8)

| | Acceleration condition (month) | | | |
|---|---|---|---|---|
| | Example No. | 0 | 3 | 6 |
| Degradation product | Example 5 | 0.031 | 0.042 | 0.072 |
| of irbesartan(%) | Comparative Example 1 | 0.031 | 0.035 | 0.062 |
| | Comparative Example 2 | 0.028 | 0.031 | 0.073 |
| | Comparative Example 3 | 0.044 | 0.073 | 0.161 |

As can be seen in Tables 6A and 6B, degradation products of atorvastatin (RRT 1.81) and irbesartan (RRT 0.8) increased in quantity with time. According to the ICH guideline for related substance, both irbesartan and atorvastatin must be degraded at a rate of 0.2% or less for 6 months in an acceleration condition or for 24 to 36 months in a long-term condition. The formulation according to the present invention was improved in stability of atorvastatin owing to the presence of the alkaline additive. With superiority in stability to the formulations of Comparative Examples 1 to 3, the formulation of the present invention was proven for the product having a shelf life of 3 or more years.

### Test Example 2

### Dissolution Assay of Irbesartan

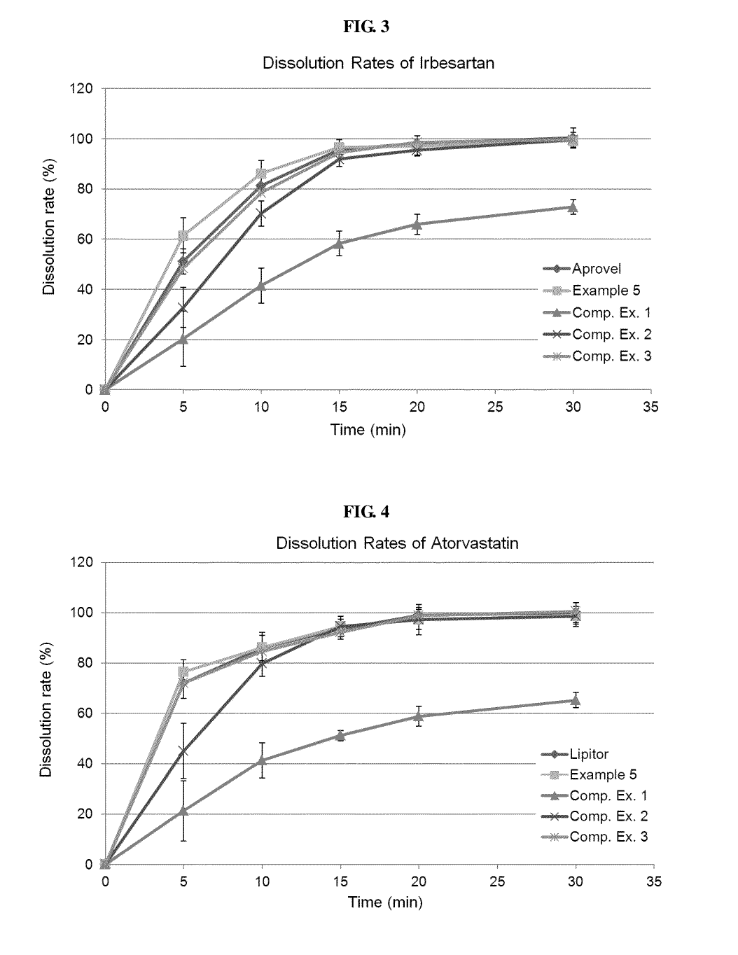

Formulations of Example 5 and Comparative Examples 1 to 3 were assayed for irbesartan dissolution in the following dissolution test condition. Aprovel™ 150 mg (Sanofi-Aventis) was used as a control. The results are shown in FIG. 3.

<Dissolution Test Condition>

(1) Dissolution tester: PTWS-1210 (Pharmatest, Germany)

(2) Dissolution medium: 0.1 mol/L HCl

(3) Temperature of medium: 37±0.5° C.

(4) Medium volume: 1000 mL

(5) Stirring speed: 50 rpm

(6) Sampling: Dissolution media were taken 5, 10, 15, 30 and 45 min after the test was conducted, and filtered through a 0.45 μm membrane filter. After sampling every time, a fresh dissolution medium was supplemented in the same volume to the tester.

<Analysis Method>

(1) Analyzer: High performance liquid chromatography (HPLC)

(2) Mobile phase: acetonitrile/tetrahydrofuran/buffer 1 (31:9:60)

(buffer 1=0.05 M NH₄H₂PO₄, pH 5.0, pH adjusted with ammonia water)

(3) Detector: UV spectrophotometer (244 nm)

(4) Column: column with an inner diameter of about 4.6 mm and a length of about 150 mm, loaded with octadecylsilylated silica gel 5 μm

(5) Flow rate: 1.8 mL/min

### Test Example 3

### Dissolution Test of Atorvastatin Calcium

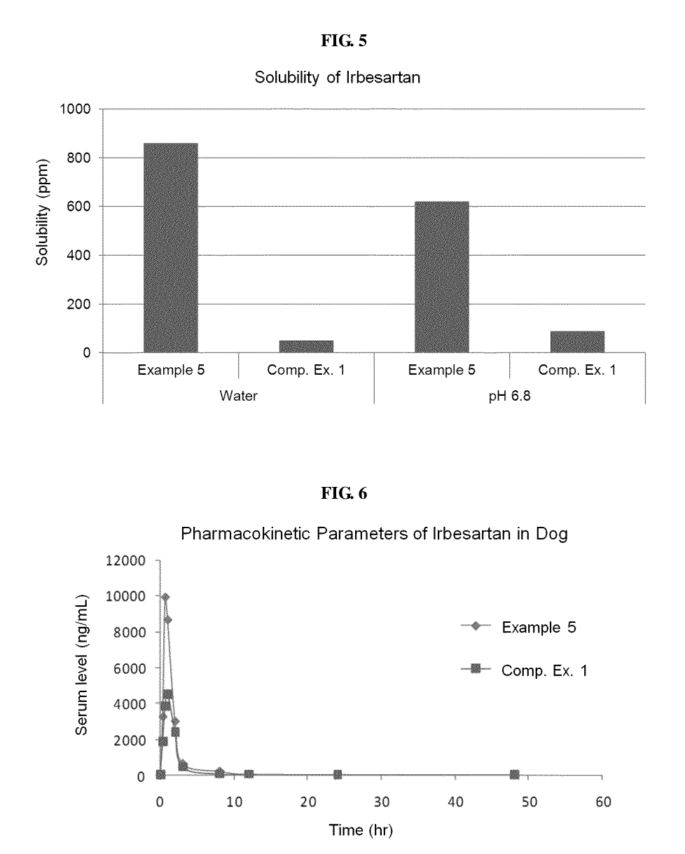

Formulations of Example 5 and Comparative Examples 1 to 3 were assayed for atorvastatin calcium dissolution in the following dissolution test condition. Lipitor™ (Pfizer) 20 mg was used as a control. The results are shown in FIG. 4.

<Dissolution Test Condition>

(1) Dissolution tester: PTWS-1210 (Pharmatest, Germany)

(2) Dissolution medium: Purified water

(3) Temperature of medium: 37±0.5° C.

(4) Medium volume: 900 mL

(5) Stirring speed: 50 rpm

(6) Sampling: Dissolution media were taken 5, 10, 15, 30 and 45 min after the test was conducted and filtered through a 0.45 μm membrane filter. After sampling every time, a fresh dissolution medium was supplemented in the same volume to the tester.

As can be seen in FIGS. 3 and 4, the capsule comprising tablets larger than 3 mm (Comparative Example 2) was low in the first 5 min dissolution rate, and exhibited a similar dissolution behavior to that of the control after 10 min, which corresponded to a lag time for which the exterior gelatin capsule was disintegrated prior to the dissolution of the tablets positioned inside the capsule, indicating that the gelatin influenced the disintegration of the tablets positioned inside the capsule. In addition, the capsule free of an alkaline stabilizer (Comparative Example 1) remained low in the dissolution rate of atorvastatin even until the late phase.

In contrast, the capsule formulation according to the present invention (Example 5) characterized by mini-tablets was disintegrated quickly. In detail, the dissolution of the active ingredients started as soon as the dissolution medium flowed into the gelatin capsule through holes generated upon the disintegration of the capsule. Due to the small size of the tablets, the active ingredients were more quickly dissolved from the capsule. In addition, the formulation of the present invention was found to have an equivalent level of dissolution rate as in the control, as analyzed for active ingredients.

Even though lower in total weight, the capsule formulation of the present invention exhibited dissolution rates at an equivalent level as in the two-layer formulation (Comparative Example 3), and thus exerted a higher dissolution effect, compared to the two-layer formulation. Accordingly, the capsule formulation of the present invention was improved in stability and dissolution rate while decreasing in excipient content, which leads to an expectation of improved drug compliance.

### Test Example 4

### Assay for Solubility of Irbesartan

The formulations prepared in Example 5 and Comparative Example 1 were assayed for the solubility of irbesartan. According to USP Dissolution Apparatus 2 (Paddle), 10 capsules of each of the samples were dissolved in 1,000 mL of water and 1,000 mL of a pH 6.8 solution, while stirring at 50 rpm. After 12 hrs, the solutions were analyzed for irbesartan solubility. The results are given in FIG. 5.

As apparent from the data of FIG. 5, the formulation of the present invention exhibited much higher irbesartan solubilities in water and a pH 6.8 solution, as compared to the alkaline additive-free formulation of Comparative Example 1. These results demonstrate that the alkaline additive improves the solubility of the water-insoluble compound irbesartan.

### Test Example 5

### Assay for Bioavailability of Irbesartan

The formulations prepared in Example 5 and Comparative Example 1 were assayed for the bioavailability of irbesartan in beagle dogs according to the experimental procedure given in Table 7, below.

TABLE 7
Assay for bioavailability of irbesartan

| | |
|---|---|
| Title | Study on in vivo pharmacokinetic behavior of irbesartan in |
| | beagle dog after single dose |
| Purpose | To evaluate the bioavailability of the irbesartan formulation |
| | improved in solubility |
| Test system | Test animal: beagle dogs |
| | Gender: male |
| | No. of test animal: 6 in each group, randomized |
| | crossover study |
| Test group | Test group: administered with the formulation of Example 5 |
| | Control: administered with the formulation of Comparative |
| | Example 1 |
| Process | 1) fasted for 12 hrs before administration, fed only with |
| | water |
| | 2) orally administered with the formulation of Example 5 or |
| | Comparative Example 1 |
| | 3) blood sampled at 0, 0.33, 0.66, 1, 2, 3, 8, 12, 24 and |
| | 48 hrs after administration |
| | 4) irbesartan levels measured using LC/MS |
| Statistics | Pharmacokinetic parameters measured using a data |
| | processing program (KE-Test). |

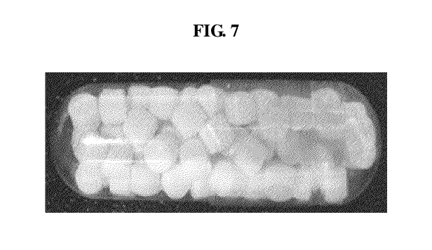

The results are given in Table 8 and FIG. 6. FIG. 6 shows arithmetic means of serum levels of irbesartan (ng/mL) versus time (hr) on a linear scale.

TABLE 8
Pharmacokinetic parameter of irbesartan

| | Irbesartan | |
|---|---|---|
| Parameter | Example 1 | Comparative Example 1 |
| AUC₀₋₄₈ (ng · hr/mL) | 20136.4 ± 4835.7 | 9956.0 ± 6859.6 |
| Cₘₐₓ (ng/mL) | 13856.4 ± 5746.5 | 6493.4 ± 3349.8 |
| Tₘₐₓ (hr) | 1.3 ± 0.7 | 0.8 ± 0.6 |

As can be seen from data of Table 8 and FIG. 6, the formulation of the present invention was higher in bioavailability than the alkaline additive-free formulation of Comparative Example 1, indicating that the alkaline additive improved the bioavailability of irbesartan.

While the invention has been described with respect to the above specific embodiments, it should be recognized that various modifications and changes may be made to the invention by those skilled in the art which also fall within the scope of the invention as defined by the appended claims.

What is claimed is:

1. A pharmaceutical composite capsule formulation comprising:
1) an independent irbesartan unit comprising irbesartan or a pharmaceutically acceptable salt thereof; and
2) an independent HMG-CoA reductase inhibitor unit comprising an HMG-CoA reductase inhibitor or a pharmaceutically acceptable salt thereof, and an alkaline additive,
wherein said independent units are separated from each other within a capsule.

2. The pharmaceutical composite capsule formulation of claim 1, wherein the HMG-CoA reductase inhibitor is selected from the group consisting of rosuvastatin, lovastatin, atorvastatin, pravastatin, fluvastatin, pitavastatin, simvastatin, rivastatin, cerivastatin, velostatin, mevastatin, a pharmaceutically acceptable salt thereof, a precursor thereof and a mixture thereof.

3. The pharmaceutical composite capsule formulation of claim 2, wherein the HMG-CoA reductase inhibitor is atorvastatin calcium.

4. The pharmaceutical composite capsule formulation of claim 1, wherein the independent irbesartan unit and the independent HMG-CoA reductase inhibitor unit are each in a granule or tablet form.

5. The pharmaceutical composite capsule formulation of claim 4, wherein at least one of the independent irbesartan unit and the independent HMG-CoA reductase inhibitor unit takes a tablet form.

6. The pharmaceutical composite capsule formulation of claim 4, wherein the tablet has a diameter of 3 mm or less.

7. The pharmaceutical composite capsule formulation of claim 4, wherein the tablet has a thickness of 3 mm or less.

8. The pharmaceutical composite capsule formulation of claim 4, wherein the tablet further comprises a coating layer.

9. The pharmaceutical composite capsule formulation of claim 8, wherein the coating layer is made of a coating material selected from the group consisting of methyl cellulose, ethyl cellulose, polyvinyl alcohol, polyvinyl pyrrolidone, hydroxyethyl cellulose, hydroxypropyl methyl cellulose and a mixture thereof.

10. The pharmaceutical composite capsule formulation of claim 9, wherein the coating material is employed in an amount of from 1 to 20 wt %, based on the total weight of the tablet.

11. The pharmaceutical composite capsule formulation of claim 1, wherein the capsule is a hard capsule.

12. The pharmaceutical composite capsule formulation of claim 11, wherein the capsule is made of a material selected from the group consisting of hypromellose, pullulan, gelatin and polyvinyl alcohol.

13. The pharmaceutical composite capsule formulation of claim 1, wherein the alkaline additive is selected from the group consisting of NaHCO₃, CaCO₃, MgCO₃, KH₂PO₄, K₂HPO₃, calcium phosphate tribasic, arginine, lysine, histidine, meglumine, aluminum magnesium silicate, aluminum magnesium metasilicate, a salt thereof and a mixture thereof.

14. The pharmaceutical composite capsule formulation of claim 13, wherein the alkaline additive is NaHCO₃, MgCO₃, or a mixture thereof.

15. The pharmaceutical composite capsule formulation of claim 1, wherein the alkaline additive is used in an amount of from 2 to 10 parts by weight, based on 1 part by weight of the HMG-CoA reductase inhibitor.

16. The pharmaceutical composite capsule formulation of claim 1, wherein the independent irbesartan unit further comprises a pharmaceutically acceptable additive selected from the group consisting of a binder, a disintegrant, a lubricant, a diluent, a colorant, an anti-tackifier, a surfactant and a mixture thereof.

17. The pharmaceutical composite capsule formulation of claim 1, comprising irbesartan or the pharmaceutically acceptable salt thereof in an amount of from 8 mg to 600 mg per unit formulation.

18. The pharmaceutical composite capsule formulation of claim 1, comprising the HMG-CoA reductase inhibitor or the pharmaceutically acceptable salt thereof in an amount of from 5 mg to 80 mg per unit formulation.

19. The pharmaceutical composite capsule formulation of claim 1, wherein the irbesartan and the HMG-CoA reductase inhibitor are individually released at a rate of 80% or higher within 30 min.

20. The pharmaceutical composite capsule formulation of claim 19, wherein the irbesartan and the HMG-CoA reductase inhibitor are individually released at a rate of 80% or higher within 15 min.

21. The pharmaceutical composite capsule formulation of claim 1, which is used for preventing or treating a disease selected from the group consisting of hypertension, hypercholesterolemia, hyperlipidemia, myocardial infarction, stroke, a disease requiring angioplasty and chronic stable angina pectoris.

22. A method for preparing the pharmaceutical composite capsule formulation of claim 1, comprising:
1) forming irbesartan granules or tablets comprising irbesartan or a pharmaceutically acceptable salt thereof;
2) forming HMG-CoA reductase inhibitor granules or tablets comprising an HMG-CoA reductase inhibitor or a pharmaceutically acceptable salt thereof, and an alkaline additive;
3) loading the irbesartan granules or tablets of step 1) and the HMG-CoA reductase inhibitor granules or tablets of step 2) into a hard capsule, such that said irbesartan granules or tablets are separated from said HMG-CoA reductase inhibitor granules or tablets within the capsule.

23. The method of claim 22, wherein at least one of the irbesartan or the pharmaceutically acceptable salt thereof in step 1) and the HMG-CoA reductase inhibitor or a pharmaceutically acceptable salt thereof in step 2) is formed into tablets.

24. The method of claim 23, further comprising coating the tablets.
